(19) United States
(12) Patent Application Publication
(10) Pub. No.: US 2026/0283992 A1
(43) Pub. Date: Sep. 24, 2026

(54) COMPOSITIONS AND METHODS OF USE OF GAMMA-KETOALDHEYDE SCAVENGERS FOR TREATING, PREVENTING OR IMPROVING FIBROSIS OF THE LIVER

(71) Applicant: MTI Biotech, Inc., Ames, IA (US)

(72) Inventors: John Rathmacher, Story City, IA (US); Naji Abumrad, Nashville, TN (US); Charles Flynn, Nashville, TN (US)

(73) Assignee: MTI Biotech, Inc., Ames, IA (US)

(21) Appl. No.: 19/687,892

(22) Filed: May 26, 2026

Related U.S. Application Data
(63) Continuation-in-part of application No. 18/213,416, filed on Jun. 23, 2023, now Pat. No. 12,636,269.
Application No. 18/213,416 is a continuation of application No. 17/064,154, filed on Oct. 6, 2020, now abandoned.
Application No. 17/064,154 is a continuation-in-part of application No. 16/122,416, filed on Sep. 5, 2018, now abandoned.
(60) Provisional application No. 62/554,294, filed on Sep. 5, 2017.

(51) Int. Cl. A61K 31/197; A61K 31/137; A61P 1/16

(52) U.S. Cl. CPC A61K 31/197; A61K 31/137; A61P 1/16

(57) ABSTRACT

Methods and compositions for use in treating, attenuating, preventing or improving liver fibrosis in a subject are described. The compounds of the present invention are gamma-ketoaldehyde scavengers.

The present application is a continuation-in-part of U.S. application Ser. No. 18/213,416, which is a continuation of U.S. application Ser. No. 17/064,154, filed Oct. 6, 2020, which is a continuation-in-part of U.S. application Ser. No. 16/122,416, filed Sep. 5, 2018, which claims the benefit of U.S. Provisional Application Ser. No. 62/554,294, filed Sep. 5, 2017, the disclosures of each of which are hereby incorporated by reference in their entireties.

The present application further incorporates subject matter from International Application WO 2020/154731 A1, claiming priority to U.S. Provisional Application Ser. No. 62/796,999, filed Jan. 25, 2019, and from US 2022/0162240 A1, claiming priority to U.S. Provisional Application Ser. No. 62/796,889, filed Jan. 25, 2019, the disclosures of each of which are hereby incorporated by reference in their entireties.

### BACKGROUND OF THE INVENTION

### 1. Field

The present invention relates to a composition comprising a gamma-ketoaldehyde scavenging compound, such as 2-Hydroxybenzylamine (2-HOBA), and methods of administering a gamma-ketoaldehyde scavenger to treat, prevent, attenuate, reduce, slow the progression of, or improve fibrosis of the liver.

### 2. Background

Liver fibrosis is a histological change caused by liver inflammation and/or chronic injury. Damage to the liver causes liver stellate cells to become overactive and triggers the extra cellular #5248770 matrix (ECM) synthesis to increase. Excess amounts of collagen fiber deposits occurs in the extracellular spaces of the liver cells which causes the liver cells to lose blood infusion and become hardened. Fibrosis is a common aspect of many liver diseases and is defined as the formation of scar tissue in the liver. Various etiologies give rise to hepatic fibrosis, including but not limited to hepatitis, nonalcoholic steatohepatitis (NASH), nonalcoholic fatty liver disease (NAFLD), toxins, alcoholic liver disease (ALD), genetic conditions, cholestatic disorders, and autoimmune diseases. Indicators of liver fibrosis included deposition of fibrotic tissue and activation of the fibrogenesis cascade. Fibrosis may produce permanent scarring of the hepatic tissue which is known as cirrhosis.

In the case of NASH, there are two hallmark histologic features: hepatic inflammation and fibrosis. While no FDA-approved therapeutics for NASH exist, several potential options have been investigated; the most promising of which include vitamin E, thiazolidinediones, and pentoxifylline. Each of these has shown some borderline clinical efficacy, but all are limited by their potential for side effects and/or toxicity, and importantly, none of these therapeutics have improved fibrosis, the strongest indicator of mortality in NASH.

γ-ketoaldehydes (γ-KA, also known as isolevuglandins or isoketals) are highly reactive lipid aldehydes that rapidly react with lysine residues and phosphatidylethanolamine to form adducts. γ-KA lipid and protein adducts have been observed in several animal models of liver disease as well as in humans with NASH. Preliminary data from humans with NASH also indicate elevated γ-KA-protein adduct formation in liver, and γ-KA-protein adducts similarly induce liver injury. γ-KA-protein adducts are linked to the loss of protein function, mitochondrial dysfunction, ER stress, and pro-inflammatory cytokine expression.

2-hydroxy-benzylamine (2-HOBA or salicylamine), a staple of buckwheat, was found to be a potent scavenger of γ-KAs scavenging γ-KAs 980-fold faster than the rate of formation of γ-KA-protein adducts. Studies have shown that 2-HOBA is 980 times more reactive than lysine with γ-KAs. Importantly, they showed that this γ-KA scavenger does not inhibit cyclooxygenase enzymes. Studies have shown that 2-HOBA dramatically protected HepG2 cells against H₂O₂-induced cytotoxicity.

It has recently been found that γKAs induced activation of human hepatic stellate cells (HSCs) to a pro-inflammatory/pro-fibrogenic phenotype. HSCs, which make up ~10% of resident liver cells, are quiescent in normal, healthy liver. However, in response to liver injury, HSCs become activated and transdifferentiate into proliferative, inflammatory myofibroblasts, which are characterized by enhanced extracellular matrix production. As such, activated HSCs are well-established as the major fibrogenic cells in the liver and are strongly implicated in the development of hepatic fibrosis in states of chronic liver injury. Oxidative stress, particularly the products of lipid oxidation, has direct pro-inflammatory/pro-fibrogenic effects on HSCs. Longato et al. recently identified γKA as novel HSC activators by exposing primary human HSC to synthetic 15-E₂-isolevuglandin (15-E2-IsoLG). Exposure to non-cytotoxic levels of 15-E₂-IsoLG promoted HSC activation, as evidenced by upregulated α-SMA expression, MAPK activation, and increased cytokine production.

Without being bound by theory or mechanism, the present inventors have discovered that selective scavengers of γKAs attenuate, reduce, treat, slow the progression of and/or improve hepatic fibrosis. Further, the compositions of the present invention do not present the adverse effects or toxicity associated with existing therapeutics for treating liver diseases such as NASH.

The isoketal scavengers of the present invention are compounds such as salicylamine (SA), for example, and analogs thereof.

The present invention includes use of gamma ketoaldehyde scavengers, including 2-HOBA, to scavenge toxic oxidized lipids (ketoaldehydes) to treat, prevent, attenuate, reduce, slow the progression of, or improve fibrosis of the liver hepatic fibrosis.

### SUMMARY OF THE INVENTION

Disclosed is a method for treating, preventing and/or attenuating hepatic fibrosis that comprises identifying a subject in need of treatment, prevention and/or attenuation of hepatic fibrosis, and administering to said subject an effective isoLG scavenging amount of at least one compound of the following formula:

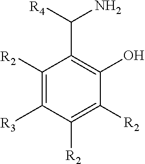

wherein R₂ is independently chosen from H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof.

In one embodiment, R₂ is independently chosen from H, ethyl, methyl. In another embodiment, the compound is 2-hydroyxbenzylamine, methyl-2-hydroyxbenzylamine, ethyl-2-hydroyxbenzylamine. In another embodiment, the compound is:

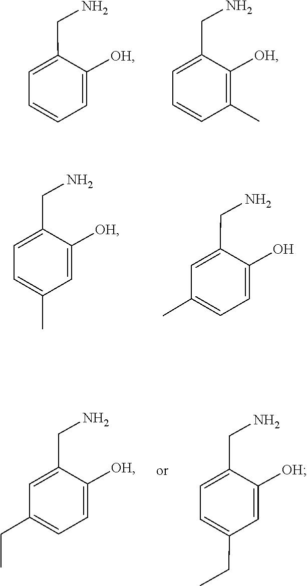

or a pharmaceutically acceptable salt thereof.

In another embodiment, the compound is:

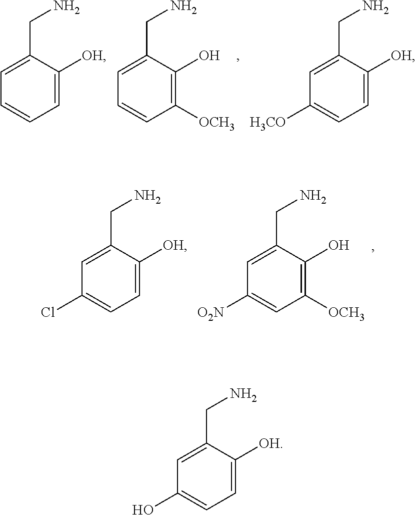

Or a pharmaceutically acceptable salt thereof.

In one embodiment, the disclosed treating step inhibits the progression of hepatic fibrosis. In one embodiment, the disclosed treating step attenuates the severity of hepatic fibrosis. Also, in one embodiment, the disclosed treating step mitigates the damaging effects of hepatic fibrosis.

In another embodiment, the compound or pharmaceutically acceptable salt thereof is administered in a composition that comprises said compound or a pharmaceutically acceptable salt thereof and a pharmaceutically acceptable carrier.

One object of the present invention is to provide compositions used treat, prevent, attenuate, reduce, slow the progression of, and/or improve hepatic fibrosis.

Another object of the present invention is to provide a therapeutic or effect amount of a preparation of the compound of the present invention to treat, prevent, attenuate, reduce, slow the progression of, or improve the symptoms of hepatic fibrosis and/or reduces the severity of hepatic fibrosis symptoms.

A further object of the present invention includes providing a novel nutritional therapy that will treat, prevent, attenuate, reduce, slow the progression of, or improve fibrosis of liver fibrosis. The nutritional therapy can be used to improve overall liver health and support healthy liver function.

An additional object of the present invention includes providing compositions and methods of use of 2-HOBA, alternatively named salicylamine, SAM, 2-hydroxylbenzylamine, and pentylpyridoxamine (PPM).

### BRIEF DESCRIPTION OF THE FIGURES

FIGS. 1a to 1b are images of slides depicting Picosirius Red staining of fibrosis in control and 2-HOBA treated mice.

FIG. 2 is a graph depicting the fibrosis score in control and 2-HOBA treated mice.

FIG. 3 depicts gene expression profiles by qRT-PCR.

### DETAILED DESCRIPTION OF THE INVENTION

All publications cited or mentioned herein are incorporated by reference to disclose and describe the methods and/or materials in connection with which the publications are cited.

The compositions described herein are used treat, prevent, attenuate, reduce, slow the progression of, and/or improve hepatic fibrosis.

A therapeutic or effect amount is a preparation of the compound of the present invention that treat, prevent, attenuate, reduce, slow the progression of, or improve the symptoms of hepatic fibrosis and/or reduces the severity of hepatic fibrosis symptoms.

The present invention includes a novel nutritional therapy that will treat, prevent, attenuate, reduce, slow the progression of, or improve fibrosis of liver fibrosis. The nutritional therapy can be used to improve overall liver health and support healthy liver function.

The present invention comprises a means to specifically prevent the formation of γKA-adducts in the liver using a class of bifunctional electrophile (BFE) “scavenger” molecules. A series of phenolic amines that includes pyridoxamine and its water soluble derivative 2-HOBA, a natural product of buckwheat seed comprise the preferred embodiment. 2-HOBA in particular reacts 980-fold faster with IsoLGs than with lysine, preventing protein and lipid adduction in vitro and in vivo.

The present invention includes compositions and methods of use of 2-HOBA, alternatively named salicylamine, SAM, 2-hydroxylbenzylamine, and pentylpyridoxamine (PPM).

In eukaryotic cells, the mitochondria are the site of oxidative phosphorylation, in this capacity they are at risk from both suffering from oxidative damage as well as generating oxidative damage. Targeting delivery of SA and SA derivatives, which exhibit antioxidant properties to the mitochondria allow the molecules to be administered at a lower dosage, thus avoiding potential adverse effects of using higher doses to achieve therapeutic outcomes, while still producing a desirable therapeutic result.

SA has a very short half-life in the blood, estimated to be about 120 minutes. In part because of its short half-life in the blood stream to maintain therapeutic levels of SA in the blood stream it may be necessary to administer SA in at least three divided doses. Moreover, in blood salicylamine appears to be processed into salicylic acid. In blood, salicylic acid has a longer half-life than salicylamine. Accordingly, repeated doses of SA over time may result in an accumulation of very high levels of salicylic acid. Furthermore, high levels of salicylic acid may modify the metabolism of SA, thereby diminishing the therapeutic effectiveness of SA.

By way of explanation and not limitation, if salicylamine acts on the mitochondria, directing a higher percentage of SA administered to a patient to the mitochondria may produce a better therapeutic outcome. Targeting the mitochondria directly by way of the TPP-SA conjugates disclosed herein reduces the length of time that SA is exposed to the blood, creates a higher effective concentration of SA at the mitochondria, and makes it possible to deliver a lower dose of the SA active agent.

There is also the possibility that the accumulation of salicylic acid in the blood could result in very high levels that are either toxic or otherwise have adverse effects. The half-life of salicylic acid in the blood is anywhere between 2 and 12 hours depending on the dose used or the amount produced. Nearly 80-90% of all salicylate in the blood is bound to protein, and the rest is in the free form, and the free form is the one detectable by normal assays. Most of the elimination of circulating salicylate is via urine. A blood level of equal to or exceeding 35 mg/dL is considered toxic, hence any further accumulation of salicylic acid in the body could become high enough to cause death. Toxicity often appears within a few days of use. The most severe cases, leading to coma and death, occurred in patients with psoriasis, who have had salicylic acid topically applied to the skin in very high doses. Accumulation of salicylic acid does not occur if the composition is targeted directly to the mitochondria, allowing for much lower dosages.

Briefly, the safe doses of SA currently taught in the art produce increased plasma levels of SA. When SA is present in the blood stream at elevated levels a portion of the blood borne SA may find its way into the cell; ultimately, a portion of the SA administered to the patient may find its way into the mitochondria.

Some strategies for preferentially or selectively delivering molecules to the mitochondria exploit the substantial negative electrochemical potential maintained across the inner mitochondrial membrane. Delocalized lipophilic cations are particularly effective at crossing the hydrophobic membranes and, hence, preferentially accumulate within the mitochondrial matrix. Some molecules, which take advantage of this approach, include a triphenylphosphonium (TPP) salt headpiece tethered to a molecule to be delivered into the mitochondria. TPP has the ability to be transported into the mitochondria against a concentration gradient. In other words, tethering a compound to TPP will result in concentrations that are much higher in the mitochondria than in the cytoplasm or the blood. In some aspects of the present disclosure described herein, the dosage of TPP-bound SA required will be much lower than SA alone, and thus mitigating any potential toxic or other unwanted side effects.

In some aspects of the present disclosure disclosed herein, salicylamine derivatives are targeted directly to the mitochondria. This class of molecules prevents the formation of γKA protein adducts by acting as a surrogate amine for adduction rather than relying upon antioxidant effects to suppress the formation of formation of γKAs. This novel set of compounds provides both a valuable tool for further probing biological pathways, as well as a potentially powerful treatment for conditions caused by inflammation and oxidative stress.

Examples of compounds of the present invention include, but are not limited to, compounds selected from the formula or analogs thereof, and pharmaceutical salts thereof:

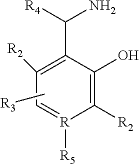

wherein:
R is Nor C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N;
and stereoisomers and analogs thereof.

Another embodiment of the present invention includes compounds of the following formula, and their use in methods for treating, preventing, or ameliorating liver fibrosis to a subject with or at risk of liver fibrosis:

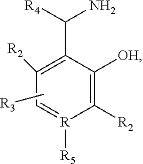

wherein:
R is N or C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof.

In certain embodiments, the compound may be selected from the compounds disclosed herein. In a preferred embodiment, the compound may be salicylamine. Other compounds that may be used include methyl-2-HOBA or ethyl-2-HOBA. The present invention includes administering to a patient in need thereof an effective amount of at least one isoLG scavenger compound of the present invention, or a pharmaceutically acceptable salt thereof.

Another embodiment of the present invention is a method for treating, preventing, or ameliorating liver fibrosis to a subject with or at risk of liver fibrosis, thereby inhibiting or treating the liver fibrosis, comprising the step of co-administering to the subject at least one compound in a dosage and amount effective to treat the dysfunction in the mammal, the compound having a structure represented by a compound of the following formula:

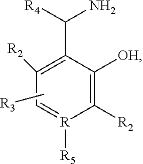

wherein:
R is N or C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; with a drug having a known side effect of treating, preventing, or ameliorating liver fibrosis.

Examples of compounds that may be used with the methods disclosed herein include, but are not limited to, compounds selected from the formula:

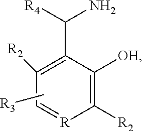

wherein:
R is N or C;
R₂ is independently H, substituted or unsubstituted alkyl;
R₃ is H, halogen, alkoxy, hydroxyl, nitro;
R₄ is H, substituted or unsubstituted alkyl, carboxyl; and pharmaceutically acceptable salts thereof.

Further examples include compounds of the following formula:

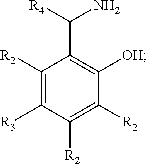

wherein: R₂ is independently chosen from H, substituted or unsubstituted alkyl; R₃ is H, halogen, alkyl, alkoxy, hydroxyl, nitro; R₄ is H, substituted or unsubstituted alkyl, carboxyl; and pharmaceutically acceptable salts thereof. In other embodiments, R₂ is independently chosen from H, ethyl, methyl.

In a preferred embodiment, the compound is salicylamine (2-hydroxybenzylamine or 2-HOBA).

The compound may be chosen from:

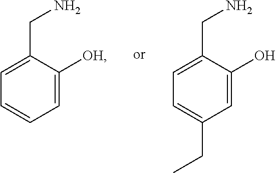

or a pharmaceutically acceptable salt thereof.

The compound may also be chosen from:

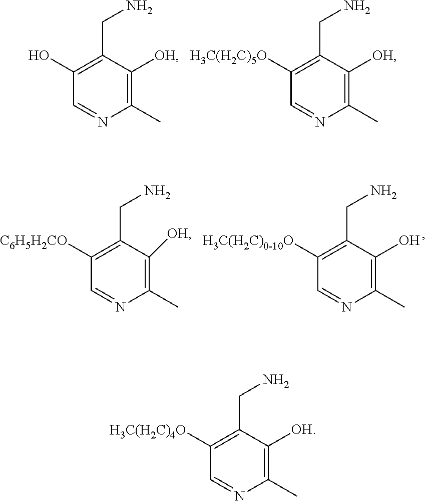

or a pharmaceutically acceptable salt thereof.

The compounds or analogs may also be chosen from:

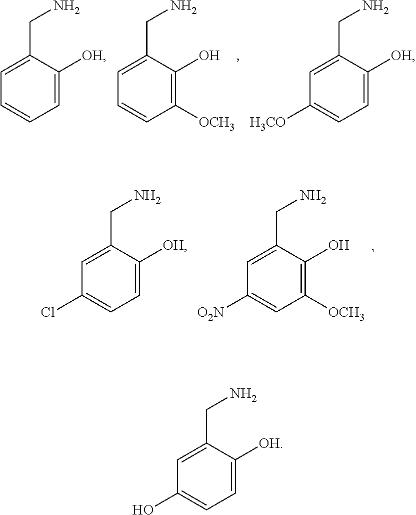

or a pharmaceutically acceptable salt thereof.

The compounds may also be chosen from:

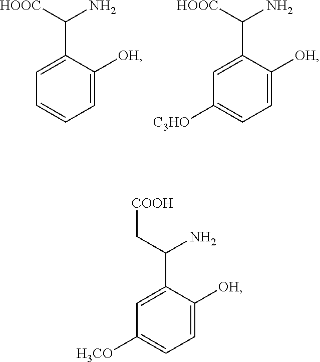

or a pharmaceutically acceptable salt thereof.

The compounds may also be chosen from

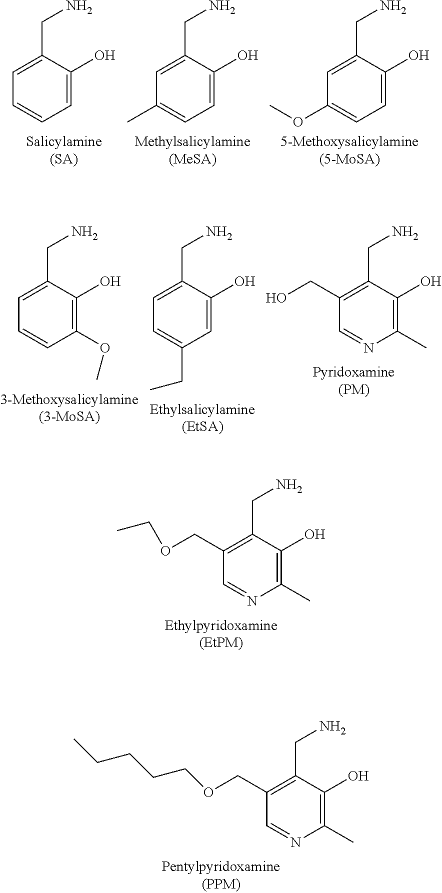

or a pharmaceutically acceptable salt thereof.

In further embodiments, the present invention comprises a compound of Formula I: wherein R1 is hydrogen with an optional counterion; R2 is selected from hydrogen or alkyl, optionally further substituted with R4; R3, which may be substituted at any ring position (e.g., ortho, meta or para to the functional groups), is either a hydrogen, hydroxy, acyl, or alkoxy, optionally substituted with R4; R4 is a cation with an optional counterion; and n is either 1 or 2. The functional group on R3 may or may not have an intervening alkyl group chain. Optionally, the substitution pattern for Formula I is as follows: R1 is hydrogen with an optional counterion; R2 is selected from hydrogen or alkyl, optionally substituted with R4; R3 is selected from hydrogen, halogen, hydroxy, acyl, alkoxy, C1-C6 alkyl optionally substituted with alkyl, alkenyl, hydroxy, acyl, or alkoxy, or carbonyl optionally substituted with hydrogen, hydroxy, acyl, alkoxy, C1-C6 alkyl, C1-C6 alkenyl, O, N, or S, optionally substituted with hydrogen, hydroxy, or C1-C6 alkyl; R4 is a cation with optional counterion; and n is either 1 or 2.

In another embodiment, R4 is a triphenylphosphonium cation or quinone-derived ammonium cation, such as, for example, [10-(4,5-dimethyl-3,6-dioxocyclohexan-1,4-dien-1-yl)decyl](tributyl)ammonium bromide. In some embodiments, the compound is (5-((2-hydroxybenzyl)amino)pentyl)triphenylphosphonium bromide. In some embodiments, the compound is (5-((2-hydroxyphenyl)amino)pentyl)triphenylphosphonium bromide. In further embodiments, the mitochondria-targeted scavenger is a compound of the following formula, wherein the 2-aminomethylphenol moiety is tethered to a triphenylphosphonium cation: wherein X is a bond, O, or CH2-; and R is C1 to C12 substituted or unsubstituted alkyl; and stereoisomers and pharmaceutical salts thereof. In some such embodiments, R is C4, C6, or C10 alkyl. In another embodiment, the mitochondria-targeted scavenger is a compound of the following formula: wherein X is a bond, O, or CH2-; R is C1 to C12 substituted or unsubstituted alkyl; and R1 is C1 to C12 substituted or unsubstituted alkyl or acetoxymethyl; and stereoisomers and pharmaceutical salts thereof. In some embodiments, the mitochondria-targeted scavenger is a dicationic compound of the following formula: wherein each R is independent and chosen from C1 to C12 substituted or unsubstituted alkyl; and each R1 is independent and chosen from C1 to C12 substituted or unsubstituted alkyl or acetoxymethyl; and stereoisomers and pharmaceutical salts thereof. Examples of specific mitochondria-targeted compounds include, but are not limited to: (4-(4-(aminomethyl)-3-hydroxyphenoxy)butyl)triphenylphosphonium chloride (mito2HOBA); (6-(4-(aminomethyl)-3-hydroxyphenoxy)hexyl)triphenylphosphonium chloride (mito2HOBA-C6); (10-(4-(aminomethyl)-3-hydroxyphenoxy)decyl)triphenylphosphonium chloride (mito2HOBA-C10); methyl 2-(3-(aminomethyl)-4-hydroxyphenyl)acetate; and acetoxymethyl 2-(3-(aminomethyl)-4-hydroxyphenyl)acetate; and stereoisomers and pharmaceutical salts thereof. These derivates of 2-aminomethylphenol can be prepared by the orthoaclylation making use of the aldol chemistry of the phenol alkoxides. Alternatively, the allyl ether of the phenol can be made and use a Claisen rearrangement to insert an allyl group at the 6-position. These new compounds include functionality to allow for the preparation of conjugates while preserving the aminophenol moiety. Still another approach is ortho-carboxylation via the alkoxide and carbon dioxide is another approach. These compounds include functionalities to allow for the preparation of conjugates while preserving the aminophenol moiety. The compounds can be conjugated with triarylphosphines (phosphonium salts) which promote reagent to localize in the mitochondria. For example, the 6-hydroxymethyl analog can be connected with a hydroxyalkyl phosphonium salt using an acid catalyst.

Starting from hydroquinone, tetradeuteration can be accomplished using deuterochloric acid in deuterium oxide and deuterated methanol. From there, formylation can be performed using triethylamine (Et₃N), magnesium chloride, and paraformaldehyde in acetonitrile. With deuterated 2,5-dihydroxybenzaldehyde XIII, the desired deuterated salt may be synthesized using the synthesis methods described above.

Detailed synthesis methods for the compounds described herein, including TPP-SA conjugates, deuterated analogs, and the mito2HOBA series of mitochondria-targeted scavengers, are disclosed in the applications incorporated by reference herein. A person of ordinary skill in the art would be able to prepare the compounds of the present invention without undue experimentation in view of the present disclosure and the references incorporated herein.

The compounds of the present invention can be administered by any method and such methods are well known to those skilled in the art and include, but are not limited to oral administration, transdermal administration, administration by inhalation, nasal administration, topical administration, intravaginal administration, ophthalmic administration, intraaural administration, intracerebral administration, rectal administration, and parenteral administration, including injectable administration such as intravenous administration, intra-arterial administration, intramuscular administration and subcutaneous administration. The compounds can be administered therapeutically, to treat an existing disease or condition, or prophylactically for the prevention of a disease or condition.

The compounds described herein may be administered by any suitable route, including orally, enterally, parentally, by inhalation or rectally in dosage unit formulations containing conventional pharmaceutically acceptable carriers, adjuvants, and vehicles, including liposomes. The term parenteral as used herein includes, subcutaneous, intravenous, intra-arterial, intramuscular, intra-sternal, intra-tendinous, intra-spinal, intra-cranial, intra-thoracic, infusion techniques, intra-cavity, enteral, or intra-peritoneal.

The compounds of the present disclosure can be administered as the sole active pharmaceutical agent, or can be used in combination with one or more other agents useful for treating or preventing various complications, such as, for example, Alzheimer's disease and other neurodegenerative diseases, hypertension, fatty liver disease, alcohol-related liver disease, chronic obstructive pulmonary disease, pulmonary hypertension, radiation-induced tissue injury, and gastroesophageal reflux disease. The compounds of the present disclosure, as the sole active pharmaceutical agent or in combination with one or more other agents, may also be used in the prevention of ischaemia reperfusion injury and to prevent cardiac dysrhythmia, for example. When administered as a combination, the therapeutic agents can be formulated as separate compositions that are given at the same time or different times, or therapeutic agents can be given as a single composition.

Although any suitable pharmaceutical medium comprising the composition can be utilized within the context of the present invention, preferably, the composition is combined with a suitable pharmaceutical carrier, such as dextrose or sucrose.

Methods of calculating the frequency by which the composition is administered are well-known in the art and any suitable frequency of administration can be used within the context of the present invention (e.g., one 6 g dose per day or two 3 g doses per day) and over any suitable time period (e.g., a single dose can be administered over a five minute time period or over a one hour time period, or, alternatively, multiple doses can be administered over an extended time period). The composition of the present invention can be administered over an extended period of time, such as weeks, months or years. The composition can be administered in individual servings comprising one or more than one doses (individual servings) per day, to make a daily serving comprising the total amount of the composition administered in a day or 24 hour period.

Any suitable dose of the present composition can be used within the context of the present invention. Methods of calculating proper doses are well known in the art.

Some aspects include therapeutic dosages of the SA-TPP conjugate, ranging from at least about 5 ug to about 1000 mg every 24 hours. In some aspects of the present disclosure, the dose of the SA-TPP conjugate administered to a subject in a given 24 hour period or its equivalent time frame is selected from the following ranges; from at least about 5 ug to about 10 ug, from at least about 10 ug to about 20 ug, from at least about 20 ug to about 40 ug, from at least about 40 ug to about 60 ug, from at least about 60 ug to about 80 ug, from at least about 80 ug to about 100 ug, from at least about 100 ug to about 120 ug, from at least about 120 ug to about 140 ug, from at least about 140 ug to about 160 ug, from at least about 160 ug to about 180 ug, from at least about 180 ug to about 200 ug.

In some aspects, the dose in a given 24 hour period is on the order of about from at least about 200 ug to about 300 ug, or from at least about 300 ug to about 400 ug, or from at least about 400 ug to about 500 ug, or from at least about 500 ug to about 600 ug, or from at least about 600 ug to about 700 ug or, from at least about 700 ug to about 800 ug, or, from at least about 800 ug to about 900 ug, or, from at least about 900 ug to about 1000 ug. In other aspects, the dose is more than 1 mg every 24 hours, in some aspects the dose per 24-hour period is from at least about 1 mg to about 10 mg, or from about 10 mg to about 25 mg, or from about 25 mg to about 50 mg, or from about 50 mg to about 75 mg, or from about 75 mg to about 100 mg.

Some aspects include therapeutic dosages of the SA-TPP conjugate, ranging from 200 ug to 100 mg every 24 hours. In some aspects of the present disclosure, the dose of the SA-TPP conjugate is 5.0 mg every 24 hours. In some aspects, the dose is 10.0 mg every 24 hours. In other aspects, the dose is 20.0 mg every 24 hours. In still other aspects, the dose is 33.0 mg every 24 hours. In other aspects, the dose is 55.0 mg every 24 hours. In yet other aspects, the dose is 82.5 mg every 24 hours.

“Treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Prevent” or “preventing” refers to averting, stalling, stopping or hindering something from happening, including by advance action. There is overlap in treating and preventing.

“Effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

“Substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

“Alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

“Cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

“Polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A1, A2, and A3 are alkyl and/or cycloalkyl groups.

The terms “amine” or “amino” as used herein are represented by a formula NA¹A²A³ where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “hydroxyl” as used herein is represented by a formula ^{~}OH.

The term “nitro” as used herein is represented by a formula ^{~}NO₂.

As described herein, oxidative stress, and particularly the products of lipid oxidation, has direct pro-inflammatory and pro-fibrogenic effects on hepatic stellate cells (HSCs). HSCs, which make up approximately 10% of resident liver cells, are quiescent in normal, healthy liver.

However, in response to liver injury, HSCs become activated and transdifferentiate into proliferative, inflammatory myofibroblasts characterized by enhanced extracellular matrix production. Activated HSCs are well-established as the major fibrogenic cells in the liver and are strongly implicated in the development of hepatic fibrosis in states of chronic liver injury. It has been discovered that γ-ketoaldehydes (γKAs) are novel activators of HSCs, as exposure to non-cytotoxic levels of synthetic 15-E2-isolevuglandin promoted HSC activation, evidenced by upregulated α-SMA expression, MAPK activation, and increased cytokine production. Salicylamine (SA, also known as 2-HOBA or 2-hydroxybenzylamine) is a potent and selective scavenger of γKAs, reacting with γKAs approximately 980-fold faster than the rate of formation of γKA-protein adducts, thereby preventing adduction to cellular proteins and lipids.

In eukaryotic cells, the mitochondria are the site of oxidative phosphorylation, and in this capacity they are a primary source of reactive oxygen species and of γKA generation. Free radical oxidation of arachidonic acid and other polyunsaturated fatty acids within the mitochondria produces highly reactive isolevuglandins (isoLG, also known as isoketals or γ-ketoaldehydes). Because mitochondria are the major source of superoxide radicals and are rich in unsaturated fatty acids, selective delivery of SA to the mitochondria of hepatic cells—including hepatocytes and hepatic stellate cells—is expected to produce a more effective therapeutic outcome for treating, preventing, attenuating, or slowing the progression of liver fibrosis. Targeting the mitochondria directly, rather than relying on systemic distribution of SA, places the active scavenging moiety at the intracellular site where γKAs are formed, thereby intercepting these reactive lipid dicarbonyls before they can adduct to mitochondrial proteins and phosphatidylethanolamine, activate CypD, or diffuse to induce HSC activation and fibrogenesis.

Triphenylphosphonium (TPP) conjugates of SA exploit the substantial negative electrochemical potential (approximately-150 mV) maintained across the inner mitochondrial membrane. Delocalized lipophilic cations such as TPP selectively accumulate within the mitochondrial matrix by more than five hundred-fold relative to the cytoplasm. TPP-SA conjugates retain the active SA pharmacophore—the 2-aminomethylphenol moiety—which acts as a surrogate amine for adduction with γKAs rather than relying upon antioxidant effects to suppress KA formation. These conjugates preserve the aminophenol functionality that confers γKA-scavenging reactivity, and are therefore understood to scavenge γKAs with the same or better efficacy as untethered SA when present at the site of γKA generation within the mitochondria.

Without being bound by theory or mechanism, directing a higher percentage of administered SA to the mitochondria of hepatic cells by means of TPP-SA conjugates is expected to provide several therapeutic advantages in the treatment of liver fibrosis: (1) a lower effective dose of the SA active agent may be required to achieve therapeutic γKA scavenging within the liver, because the conjugate concentrates preferentially in the mitochondrial matrix; (2) reduced dosing frequency may be achieved because the mitochondria-targeted compound accumulates at the site of action rather than being rapidly cleared from the blood; (3) limited accumulation of salicylic acid may result, because targeting the mitochondria directly reduces the length of time that SA is exposed to the bloodstream and thus diminishes metabolism to salicylic acid, mitigating potential toxicity associated with elevated salicylic acid levels; and (4) a higher ratio of active SA reaches the intracellular site of γKA formation within hepatocytes and HSCs, producing greater scavenging efficiency relative to untargeted systemic SA administration.

### EXPERIMENTAL EXAMPLES

### Example 1

DIAMOND (Diet Induced Animal Model of Non-alcoholic fatty liver Disease) is a proprietary isogenic mouse strain that sequentially develops non-alcoholic fatty liver disease, non-alcoholic steatohepatitis, fibrosis, and hepatocellular carcinoma in response to a high-fat, high-sugar diet. Disease progression in the DIAMOND mice uniquely parallels human disease progression, including histopathology.

Twelve 8-wk old male DIAMOND mice were placed on ad libitum high fat diet (Harlan-ENVIGO TD.88317) and water containing glucose (18.9% w/v) and fructose (23.1% w/v); all mice remained on this diet throughout the study protocol. At 12 weeks of age, mice were divided into two groups: 1) 2-HOBA (n=6), and 2) vehicle controls (n=6). Animals in the 2-HOBA group received 2-HOBA in drinking water (1 g/L water with glucose and fructose). The vehicle control group received water without 2-HOBA (with glucose and fructose). Body weight and food intake were measured weekly. At ~23 weeks of age, all animals underwent a glucose tolerance test (GTT) and MRI imaging to assess hepatic fat. For the GTT, animals were fasted for 12 hours and then glucose (2 g/kg bw of a 100 mg/mL glucose in sterile water) was administered by oral gavage. Blood was sampled at 0, 15, 30, 45, 60, 90, and 120 minutes after glucose administration and area under the curve was calculated. Animals were sacrificed at 24 weeks of age (12 weeks of 2-HOBA or vehicle treatment). Tissues and serum were collected for analysis.

Liver sections were stained with hematoxylin and eosin (for scoring of steatosis, hepatocyte ballooning, and inflammation) and Sirius red (for assessment of fibrosis). Scoring was performed in a blinded manner for steatosis, ballooning, inflammation, and necrosis using the following criteria¹, Steatosis (0-4): 0=<5%; 1=5-25%; 2=25-50%; 3=50-75%; 4=75-100%. Ballooning (0-3): 0=absent; 1=mild (focal involving fewer than three hepatocytes); 2=moderate (focal involving more than three hepatocytes or multifocal); 3=prominent (multifocal with more than two foci of three or more hepatocytes). Inflammation (0-4): 0=absent; 1=minimal (zero to one focus per 20× field); 2=mild (two foci); 3=moderate (three foci); 4=severe (four or more foci). Serum levels of glucose, alanine transaminase, and aspartate transaminase were measured. Liver mRNA expression was assessed via RT-qPCR for the following genes: Tnfa, Nlrp1a, Il1b, Il18, Timp1, Colla1, ProCard, Nlrp3, Casp1, ProIl1b, Tgfb1, Bambi, Pdk4, and Gapdh. Two-tailed independent samples t-tests were used to compare endpoints between 2-HOBA and vehicle treated groups. Significance was set at α=0.05.

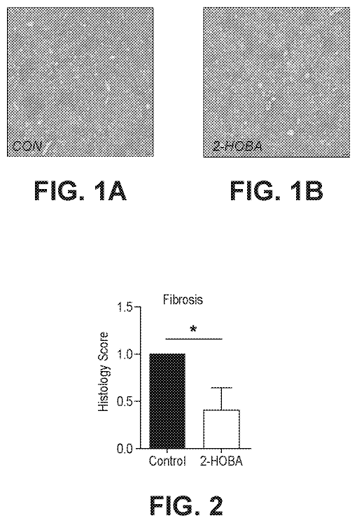

FIG. 1a-b shows Picosirius Red staining of control and 2-HOBA treated DIAMOND mouse livers. Scoring was defined on a scale of 0 to 4. All (4 out of 4) untreated mice had a fibrosis score of 1. Three of the 2-HOBA treated mice had a score of 0, while the remaining two had a score of 1.

FIG. 2 shows the fibrosis score in control and 2-HOBA treated DIAMOND mice. Despite similar degrees of hepatic steatosis and hepatocellular ballooning, the incidence of fibrosis was significantly lower in 2-HOBA compared to vehicle treated DIAMOND mice (p=0.03).

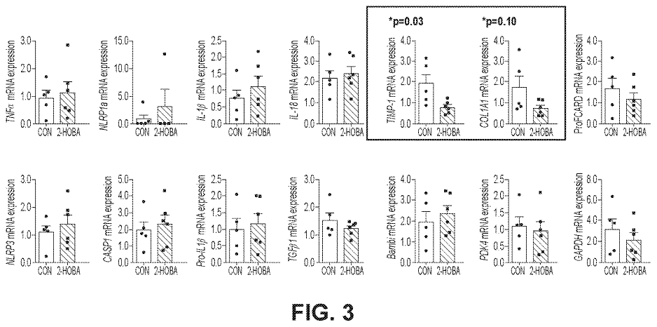

FIG. 3 shows gene expression profiles by qRT-PCR, including measurements of key genes in hepatic inflammation and fibrosis progression. Elevated levels of tissue inhibitors of metalloproteinases (TIMP) inhibit metalloproteinases (MMP) which allows extracellular matrix proteins, such as collagens, to accumulate in liver tissue. 2-HOBA reduced liver Timp1 mRNA expression in DIAMOND mice, explaining the observed beneficial effect of 2-HOBA on fibrosis development. Further, Colla1 mRNA expression levels tended to be lower. This difference was not statistically significant (p=0.08).

The observed beneficial effects of 2-HOBA on liver fibrosis is unexpected and surprising as many NASH therapeutics have failed to improve fibrosis severity. Liver fibrosis severity is the only NASH factor that independently predicts liver-related morbidity and mortality, thus therapeutics capable of preventing or attenuating fibrosis development may dramatically improve outcomes in patients with NASH. The mechanism by which 2-HOBA is thought to be therapeutic for NASH is through the attenuation of inflammatory changes in the liver. Fibrosis, however, is a secondary stage pathogenesis with a different pathogenic mechanism. 2-HOBA independently attenuates hepatic fibrosis in the DIAMOND mice without altering markers of inflammation. As such, the results described herein are unexpected and surprising.

### Example 2

γ-KAs induce activation of hepatic stellate cells (HSCs), which are the primary drivers of hepatic fibrosis. Preventing the activation of HSCs to a pro-inflammatory/pro-fibrogenic phenotype could inhibit the development of fibrosis in the liver. As transformation of HSCs into myofibroblast-like cells is considered essential for hepatic fibrosis, HSC activation will be measured using desmin, a marker of HSCs, and α-smooth muscle actin (SMA), a marker of activated HSCs, by immunohistochemistry on fixed liver sections.

Experimental Design: All experiments will be performed on 24-h-serum-starved HSCs. To prevent γKA adduction to culture media components, experimental treatments will be initiated in amino-acid and lipid-free Hank's Buffered Salt Solution for the first 15 min of exposure. This exposure duration has previously been determined to be well-tolerated by human HSCs. Human HSCs will be pre-incubated with multiple doses (1-500 μM) of 2-HOBA or vehicle before being exposed to 0.5 μM 15-E2-IsoLG. Time course experiments with 2-HOBA and 15-E₂-levuglandin will be performed to determine the optimal durations for pre-treatment and 15-E2-IsoLG exposure. Following 15-E2-IsoLG exposure, media will be collected and cells will be washed and scraped for mRNA and protein analyses. Separate replicate plates will be prepared for ROS measurements.

Human HSCs: Human stellate cells will be obtained from ZenBio (Research Triangle Park, NC) and cultured in HSC complete medium (Iscove's Modified DMEM supplemented with 20% fetal bovine serum, 2 mM glutamine, 1× non-essential amino acids, 1 mM sodium pyruvate, and 1× antibiotic-antimycotic). All experiments will be performed on cells between passage 3 and 5.

15-E₂-isolevuglandin: Synthetic 15-E2-IsoLG in DMSO will be synthesized as previously described by our consultant.

Endpoints: RNA: The expression of selected transcripts related to fibrogenic activation, cytokine production, and adhesion molecules will be measured using RT² Profiler™ PCR Arrays (Qiagen, Frederick, MD) and single-gene probe-based qRT-PCR gene expression assays, as appropriate. Protein: Immunoblot analyses will be used to measure the content and activation status of key cell signaling pathways (ERK1/2, JNK, NFκB, and p38 MAPK). Cytokines: Inflammatory cytokine concentrations will be determined in media collected after incubation with 15-E2-IsoLG and 2-HOBA. ROS/RNS: Intracellular ROS/RNS formation will be measured using the 5- (and -6-)-carboxy-2′-7′-dichlorodihydrofluorescein diacetate (Carboxy-H₂) fluorescent probe (ThermoFisher Scientific). Total cell distribution will be visualized by staining nuclei with Hoechst 33342. Images will be acquired via fluorescence microscope.

Statistics: All experiments will be performed in triplicate. Data will be analyzed by one-way (dose) or two-way (dose×time) ANOVA (as appropriate for the design), with Bonferroni's multiple comparisons tests.

### REFERENCES

1 Tilg, H. & Moschen, A. R. Evolution of inflammation in nonalcoholic fatty liver disease: the multiple parallel hits hypothesis. Hepatology 52, 1836-1846, doi: 10.1002/hep.24001 (2010).
2 Brame, C. J., Salomon, R. G., Morrow, J. D. & Roberts, L. J. Identification of extremely reactive gamma-ketoaldehydes (isolevuglandins) as products of the isoprostane pathway and characterization of their lysyl protein adducts. J. Biol. Chem 274, 13139-13146 (1999).
3 Li, W. et al. Isolevuglandins covalently modify phosphatidylethanolamines in vivo: detection and quantitative analysis of hydroxylactam adducts. Free Radic. Biol. Med 47, 1539-1552 (2009).
4 Roychowdhury, S. et al. Formation of gamma-ketoaldehyde-protein adducts during ethanol-induced liver injury in mice. Free Radic. Biol. Med 47, 1526-1538 (2009).
5 Li, X. et al. Endoplasmic reticulum stress is the crossroads of autophagy, inflammation, and apoptosis signaling pathways and participates in liver fibrosis. Inflamm Res 64, 1-7, doi: 10.1007/s00011-014-0772-y (2015).
6 Konishi, M. et al. Increased lipid peroxidation in patients with non-alcoholic fatty liver disease and chronic hepatitis C as measured by the plasma level of 8-isoprostane. J Gastroenterol. Hepatol 21, 1821-1825 (2006).
7 Davies, S. S. et al. Effects of reactive gamma-ketoaldehydes formed by the isoprostane pathway (isoketals) and cyclooxygenase pathway (levuglandins) on proteasome function. FASEB J 16, 715-717 (2002).
8 Guo, L. et al. Phosphatidylethanolamines modified by gamma-ketoaldehyde (gammaKA) induce endoplasmic reticulum stress and endothelial activation. J. Biol. Chem 286, 18170-18180 (2011).
9 Stavrovskaya, I. G. et al. Reactive gamma-ketoaldehydes formed via the isoprostane pathway disrupt mitochondrial respiration and calcium homeostasis. Free Radic. Biol. Med 49, 567-579 (2010).
10 Mont, S. et al. Accumulation of isolevuglandin-modified protein in normal and fibrotic lung. Sci. Rep 6, 24919 (2016).
11 Longato, L. et al. Reactive gamma-ketoaldehydes as novel activators of hepatic stellate cells in vitro. Free Radic Biol Med 102, 162-173, doi: 10.1016/j.freeradbiomed.2016.11.036 (2017).
12 Estes, C., Razavi, H., Loomba, R., Younossi, Z. & Sanyal, A. J. Modeling the epidemic of nonalcoholic fatty liver disease demonstrates an exponential increase in burden of disease. Hepatology, doi: 10.1002/hep.29466 (2017).
13 Wadden, T. A. et al. A two-year randomized trial of obesity treatment in primary care practice. N Engl J Med 365, 1969-1979, doi: 10.1056/NEJMoa1109220 (2011).
14 Browning, J. D. et al. Prevalence of hepatic steatosis in an urban population in the United States: impact of ethnicity. Hepatology 40, 1387-1395, doi: 10.1002/hep.20466 (2004).
Williams, C. D. et al. Prevalence of nonalcoholic fatty liver disease and nonalcoholic steatohepatitis among a largely middle-aged population utilizing ultrasound and liver biopsy: a prospective study. Gastroenterology 140, 124-131, doi: 10.1053/j.gastro.2010.09.038 (2011).
16 Kim, C. H. & Younossi, Z. M. Nonalcoholic fatty liver disease: a manifestation of the metabolic syndrome. Cleve. Clin. J. Med 75, 721-728 (2008).
17 Pagano, G. et al. Nonalcoholic steatohepatitis, insulin resistance, and metabolic syndrome: further evidence for an etiologic association. Hepatology 35, 367-372 (2002).
18 Karlas, T., Wiegand, J. & Berg, T. Gastrointestinal complications of obesity: non-alcoholic fatty liver disease (NAFLD) and its sequelae. Best Pract Res Clin Endocrinol Metab 27, 195-208, doi: 10.1016/j.beem.2013.02.002 (2013).
19 Ratziu, V., Bellentani, S., Cortez-Pinto, H., Day, C. & Marchesini, G. A position statement on NAFLD/NASH based on the EASL 2009 special conference. J Hepatol 53, 372-384, doi: 10.1016/j.jhep.2010.04.008 (2010).
20 Charlton, M. R. et al. Frequency and outcomes of liver transplantation for nonalcoholic steatohepatitis in the United States. Gastroenterology 141, 1249-1253 (2011).
21 Fujii, M. et al. A murine model for non-alcoholic steatohepatitis showing evidence of association between diabetes and hepatocellular carcinoma. Med. Mol. Morphol 46, 141-152 (2013).
22 Asgharpour, A. et al. A diet-induced animal model of non-alcoholic fatty liver disease and hepatocellular cancer. J Hepatol 65, 579-588, doi: 10.1016/j.jhep.2016.05.005 (2016).
23 Iyer, R. S., Ghosh, S. & Salomon, R. G. Levuglandin E2 crosslinks proteins. Prostaglandins 37, 471-480 (1989).
24 Murthi, K. K., Friedman, L. R., Oleinick, N. L. & Salomon, R. G. Formation of DNA-protein cross-links in mammalian cells by levuglandin E2. Biochemistry 32, 4090-4097 (1993).
25 Morrow, J. D. et al. A series of prostaglandin F2-like compounds are produced in vivo in humans by a non-cyclooxygenase, free radical-catalyzed mechanism. Proc. Natl. Acad. Sci. U.S.A 87, 9383-9387 (1990).
26 Salomon, R. G. & Miller, D. B. Levuglandins: isolation, characterization, and total synthesis of new secoprostanoid products from prostaglandin endoperoxides. Adv. Prostaglandin Thromboxane Leukot. Res 15, 323-326 (1985).
27 Bernoud-Hubac, N. et al. Low concentrations of reactive gamma-ketoaldehydes prime thromboxane-dependent human platelet aggregation via p38-MAPK activation. Biochim. Biophys. Acta 1791, 307-313 (2009).
28 Sullivan, C. B., Matafonova, E., Roberts, L. J., Amarnath, V. & Davies, S. S. Isoketals form cytotoxic phosphatidylethanolamine adducts in cells. J. Lipid Res 51, 999-1009 (2010).
29 Haukeland, J. W. et al. Systemic inflammation in nonalcoholic fatty liver disease is characterized by elevated levels of CCL2. J. Hepatol 44, 1167-1174 (2006).
30 Kojima, H. et al. Mitochondrial abnormality and oxidative stress in nonalcoholic steatohepatitis. Alcohol Clin. Exp. Res 31, S61-S66 (2007).
31 Elizondo, A. et al. Effects of weight loss on liver and erythrocyte polyunsaturated fatty acid pattern and oxidative stress status in obese patients with non-alcoholic fatty liver disease. Biol. Res 41, 59-68 (2008).
32 Wake, K. “Sternzellen” in the liver: perisinusoidal cells with special reference to storage of vitamin A. Am J Anat 132, 429-462, doi: 10.1002/aja.1001320404 (1971).
33 Puche, J. E., Saiman, Y. & Friedman, S. L. Hepatic stellate cells and liver fibrosis. Compr Physiol 3, 1473-1492, doi: 10.1002/cphy.c120035 (2013).
34 Marra, F. et al. Expression of monocyte chemotactic protein-1 precedes monocyte recruitment in a rat model of acute liver injury, and is modulated by vitamin E. J Investig Med 47, 66-75 (1999).
35 Parola, M. et al. Stimulation of lipid peroxidation or 4-hydroxynonenal treatment increases procollagen alpha 1 (I) gene expression in human liver fat-storing cells. Biochem Biophys Res Commun 194, 1044-1050, doi: 10.1006/bbrc. 1993.1927 (1993).
36 Parola, M. et al. HNE interacts directly with JNK isoforms in human hepatic stellate cells. J Clin Invest 102, 1942-1950, doi: 10.1172/JCI1413 (1998).
37 Zamara, E. et al. 4-Hydroxynonenal as a selective pro-fibrogenic stimulus for activated human hepatic stellate cells. J Hepatol 40, 60-68 (2004).
38 Amarnath, V., Amarnath, K., Amarnath, K., Davies, S. & Roberts, L. J. Pyridoxamine: an extremely potent scavenger of 1,4-dicarbonyls. Chem Res. Toxicol 17, 410-415 (2004).
39 Davies, S. S. et al. Pyridoxamine analogues scavenge lipid-derived gamma-ketoaldehydes and protect against H2O2-mediated cytotoxicity. Biochemistry 45, 15756-15767 (2006).
40 Hagstrom, H. et al. Fibrosis stage but not NASH predicts mortality and time to development of severe liver disease in biopsy-proven NAFLD. J Hepatol, doi: 10.1016/j.jhep.2017.07.027 (2017).
41 Neuschwander-Tetri, B. A. et al. Farnesoid X nuclear receptor ligand obeticholic acid for non-cirrhotic, non-alcoholic steatohepatitis (FLINT): a multicentre, randomised, placebo-controlled trial. Lancet 385, 956-965, doi: 10.1016/S0140-6736 (14) 61933-4 (2015).
42 Zagol-Ikapitte, I. A. et al. Determination of the Pharmacokinetics and Oral Bioavailability of Salicylamine, a Potent gamma-Ketoaldehyde Scavenger, by LC/MS/MS. Pharmaceutics 2, 18-29 (2010).
43 Kleiner, D. E. et al. Design and validation of a histological scoring system for nonalcoholic fatty liver disease. Hepatology 41, 1313-1321 (2005).
44 Amarnath, V., Amarnath, K., Masterson, T., Davies, S. & Roberts, L. J. A Simplified Synthesis of the Diastereomers of Levuglandin E2. Synthetic Communications 35, 397-408, doi: 10.1081/SCC-200048945 (2005).

We claim:

1. A method for treating hepatic fibrosis in a subject in need thereof, comprising:
identifying a subject in need of treatment for hepatic fibrosis;
administering to said subject an effective amount of at least one compound of the following 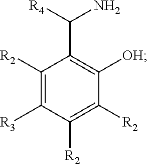
wherein:
R₂ is independently chosen from H, substituted or unsubstituted alkyl;
R₃ is H, halogen, alkyl, alkoxy, hydroxyl, nitro;
R₄ is H, substituted or unsubstituted alkyl, carboxyl; and pharmaceutically acceptable salts thereof;
wherein administration of the compound reduces liver Timp1 mRNA expression and attenuates hepatic fibrosis independent of reductions in hepatic inflammation.

2. The method of claim 1, wherein R₂ is independently chosen from H, ethyl, methyl.

3. The method of claim 1, wherein the compound is 2-hydroxybenzylamine, methyl-2-hydroxybenzylamine, ethyl-2-hydroxybenzylamine.

4. The method of claim 1, wherein the compound is: 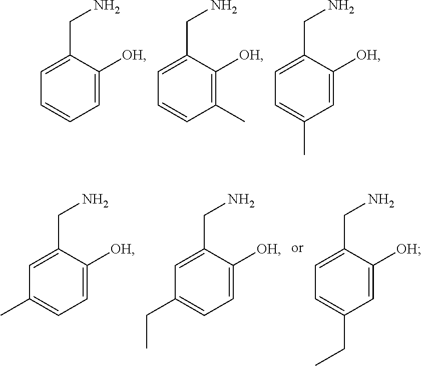
or a pharmaceutically acceptable salt thereof.

5. The method of claim 1, wherein the compound is: 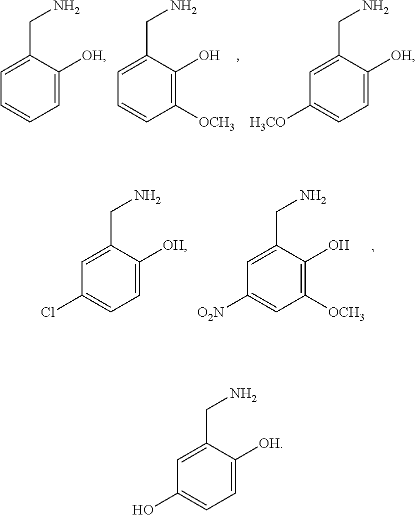
or a pharmaceutically acceptable salt thereof.

6. The method of claim 1, wherein the compound is: 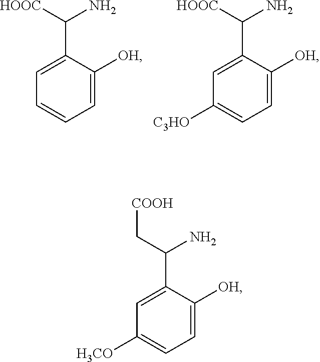
or a pharmaceutically acceptable salt thereof.

7. The method of claim 1, wherein the treating step inhibits the progression of hepatic fibrosis.

8. The method of claim 1, wherein the treating step attenuates the severity of hepatic fibrosis.

9. The method of claim 1, wherein the treating step mitigates the damaging effects of hepatic fibrosis in the subject.

10. The method of claim 1, wherein the compound or pharmaceutically acceptable salt thereof is administered in a composition that comprises said compound or a pharmaceutically acceptable salt thereof and a pharmaceutically acceptable carrier.

11. The method of claim 1, wherein the subject in need of treatment for hepatic fibrosis has or is at risk of developing hepatic fibrosis due to an etiology unrelated to alcohol consumption.

12. The method of claim 11, wherein the etiology is selected from the list consisting of nonalcoholic fatty liver disease (NAFLD), hepatitis, toxins, genetic conditions, autoimmune diseases and nonalcoholic steatohepatitis (NASH).

13. A method for treating hepatic fibrosis, comprising:
identifying a subject in need of treatment for hepatic fibrosis;
administering to said subject an effective amount of at least one mitochondria-targeted compound comprising a 2-aminomethylphenol moiety tethered to a triphenylphosphonium cation, or a pharmaceutically acceptable salt thereof, to treat the hepatic fibrosis.

14. The method of claim 13, wherein the mitochondria-targeted compound has the following formula: wherein X is a bond, O, or CH2-; and R is C1 to C12 substituted or unsubstituted alkyl; and stereoisomers and pharmaceutical salts thereof.

15. The method of claim 13, wherein the mitochondria-targeted compound is selected from the group consisting of: (4-(4-(aminomethyl)-3-hydroxyphenoxy)butyl)triphenylphosphonium chloride (mito2HOBA); (6-(4-(aminomethyl)-3-hydroxyphenoxy)hexyl)triphenylphosphonium chloride (mito2HOBA-C6); (10-(4-(aminomethyl)-3-hydroxyphenoxy)decyl)triphenylphosphonium chloride (mito2HOBA-C10); and stereoisomers and pharmaceutical salts thereof.

16. The method of claim 13, wherein the mitochondria-targeted compound is administered in a therapeutic dosage ranging from at least about 5 ug to about 1000 mg every 24 hours.
